(19) United States
(12) Patent Application Publication
(10) Pub. No.: US 2017/0273656 A1
(43) Pub. Date: Sep. 28, 2017

(54) RADIATION TOMOGRAPHIC IMAGING APPARATUS, AND PROGRAM FOR CONTROLLING THE SAME

(71) Applicant: General Electric Company, Schenectady, NY (US)

(72) Inventor: Akira Hagiwara, Tokyo (JP)

(21) Appl. No.: 15/471,384

(22) Filed: Mar. 28, 2017

(30) Foreign Application Priority Data
Mar. 28, 2016 (JP) 2016-063470

(51) Int. Cl. A61B 6/00; A61B 6/02; A61B 6/03

(52) U.S. Cl. CPC A61B 6/541; A61B 6/503; A61B 6/032; A61B 6/027; A61B 6/461

(57) ABSTRACT

A radiation tomographic imaging apparatus is characterized in comprising: a first reconstructing section for reconstructing a plurality of temporally different first radiation tomographic images for a required slice position; an information-on-movement acquiring section for acquiring information on movement of a body part in a subject based on the plurality of first radiation tomographic images; an information creating section for creating a motion profile MP indicating a temporal change of the information on movement; an identifying section for identifying a time Ts when motion of the body part in the subject stops based on the motion profile MP; and a second reconstructing section for reconstructing a second radiation tomographic image for the subject at the time Ts.

### BACKGROUND

The present invention relates to a radiation tomographic imaging apparatus for producing a radiation tomographic image of a body part moving in a subject, such as a heart, for example, and a program for controlling the radiation tomographic imaging apparatus.

In performing imaging of a heart in a radiation tomographic imaging apparatus, an EKG signal (ECG signal) is employed for image reconstruction of a radiation tomographic image using projection data collected in diastole or systole in which motion of the heart momentarily stops, as disclosed in Patent Document 1, for example (an electrocardiography-gated reconstruction method).

### SUMMARY OF INVENTION

In the electrocardiography-gated reconstruction method employing an EKG signal, however, an apparatus or several settings for acquiring the EKG signal is required. Accordingly, the inventor of the present application has made a study of producing a radiation tomographic image of a heart without using an EKG signal.

Suppose here that a radiation tomographic image is to be produced in cardiac diastole or systole without using an EKG signal, it is necessary to produce a large number of radiation tomographic images and choose images with small motion from among them because the cardiac cycle is unknown. Accordingly, in producing a radiation tomographic image of a body part moving in a subject, such as a heart, at a time when it momentarily stops or its motion is small without using an EKG signal, it is desired to suppress the number of radiation tomographic images to produce.

The invention made for solving the aforementioned problem is a radiation tomographic imaging apparatus characterized in comprising: a first reconstructing section for reconstructing a plurality of temporally different first radiation tomographic images for a required slice position in a subject based on data obtained by scanning a required range in said subject in its body-axis direction with radiation; an information-on-movement acquiring section for acquiring information on movement of a body part in said subject in said required range based on said plurality of first radiation tomographic images; information creating section for creating information on a temporal change indicating a temporal change of said information on movement acquired by said information-on-movement acquiring section; an identifying section for identifying a time when motion of said body part in said subject stops or said motion is smaller than a predetermined amount based on said information on a temporal change; and a second reconstructing section for reconstructing a second radiation tomographic image for said subject at said time based on said data.

According to the invention in the aspect described above, information on movement of a body part in a subject is detected based on a plurality of temporally different first radiation tomographic images at a required slice position, and information on a temporal change indicating a temporal change of the information on movement is created. Then, a time at which motion of the body part in the subject stops or is smaller than a predetermined amount is identified based on the information on a temporal change, and the second radiation tomographic image described above at that time is reconstructed; hence, a radiation tomographic image at a time when motion stops or is small may be obtained without using an EKG signal while suppressing the number of radiation tomographic images to produce.

### BRIEF DESCRIPTION OF DRAWINGS

FIG. 1 A diagram schematically showing a hardware configuration of an X-ray CT apparatus in accordance with an embodiment.

FIG. 2 A functional block diagram of an operation console in the X-ray CT apparatus shown in FIG. 1.

FIG. 3 A flow chart showing the flow of processing in the X-ray CT apparatus in accordance with the embodiment.

FIG. 4 A flow chart showing the flow of processing of creating information indicating a temporal change of motion of a heart.

FIG. 5 A diagram showing a relationship between a time and a position during common imaging according to a helical scan.

FIG. 6 A diagram showing a relationship between a time and a position during cardiac imaging according to a helical scan.

FIG. 7 A diagram showing the concept of reconstructing an image with a margin of scan data temporally shifted.

FIG. 8 A diagram showing a relationship between a position of a helical image and a position of an X-ray detector during data collection when a time shift is applied, and a temporal position of a weighting function and a profile thereof.

FIG. 9 A diagram showing an example of a modified profile of the weighting function when a time shift is applied.

FIG. 10 A diagram explaining calculation of a first total sum and a second total sum.

FIG. 11 A diagram showing an exemplary motion profile created for a certain slice position.

FIG. 12 A diagram explaining identification of diastole and systole.

FIG. 13 A diagram explaining reconstruction of a second X-ray tomographic image.

FIG. 14 A diagram showing local images in which motion of the heart is detected.

FIG. 15 A diagram showing an exemplary image indicating a temporal difference in motion of the heart.

FIG. 16 A diagram showing a plurality of motion profiles having mutually different phases.

FIG. 17 A diagram showing another example of the weighting function.

FIG. 18 A diagram showing another example of the weighting function.

### DETAILED DESCRIPTION

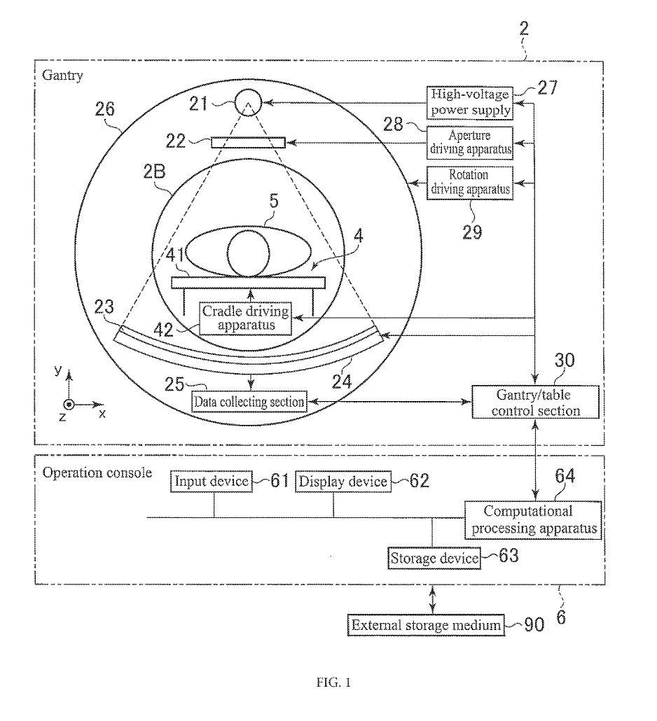

Now an embodiment of the invention will be described hereinbelow. FIG. 1 shows an X-ray CT apparatus 1, which is an exemplary embodiment of the radiation tomographic imaging system in the present invention. As shown in FIG. 1, the X-ray CT apparatus 1 comprises a gantry 2, an imaging table 4, and an operation console 6.

The gantry 2 has an X-ray tube 21, an aperture 22, a collimator device 23, an X-ray detector 24, a data collecting section 25, a rotating section 26, a high-voltage power source 27, an aperture driving apparatus 28, a rotation driving apparatus 29, and a gantry/table control section 30.

The X-ray tube 21 and X-ray detector 24 are disposed to face each other across a bore 2B.

The aperture 22 is disposed between the X-ray tube 21 and bore 2B. It shapes X-rays emitted from an X-ray focus of the X-ray tube 21 toward the X-ray detector 24 into a fan beam or a cone beam.

The collimator device 23 is disposed between the bore 2B and X-ray detector 24. The collimator device 23 removes scatter rays that would otherwise enter the X-ray detector 24.

The X-ray detector 24 has a plurality of X-ray detector elements two-dimensionally arranged in a direction of the span (referred to as channel direction) and a direction of the thickness (referred to as a row direction) of the fan-shaped X-ray beam emitted from the X-ray tube 21. Each respective X-ray detector element detects X-rays passing through a subject 5 laid in the bore 2B, and outputs an electric signal according to the intensity thereof. The subject 5 is an animate being, such as, for example, a human or an animal.

The data collecting section 25 receives the electric signal output from each X-ray detector element in the X-ray detector 24, and converts it into X-ray data for collection.

The rotating section 26 is rotatably supported around the bore 2B. The rotating section 26 has the X-ray tube 21, aperture 22, collimator device 23, X-ray detector 24, and data collecting section 25 mounted thereon.

The imaging table 4 has a cradle 41 and a cradle driving apparatus 42. The subject 5 is laid on the cradle 41. The cradle driving apparatus 42 moves the cradle 41 into/out of the bore 2B, i.e., an imaging volume, in the gantry 2.

The high-voltage power source 27 supplies high voltage and current to the X-ray tube 21.

The aperture driving apparatus 28 drives the aperture 22 and modifies the shape of its opening.

The rotation driving apparatus 29 rotationally drives the rotating section 26.

The gantry/table control section 30 controls several apparatuses and sections in the gantry 2, the imaging table 4, and the like.

The operation console 6 accepts several kinds of operation from an operator. The operation console 6 has an input device 61, a display device 62, a storage device 63, and a computational processing apparatus 64. In the present embodiment, the operation console 6 is constructed from a computer.

The input device 61 is configured to include a button, a keyboard, etc. for accepting an input of a command and information from the operator, and to further include a pointing device, and the like. The display device 62 is an LCD (Liquid Crystal Display), an organic EL (Electro-Luminescence) display, or the like.

The storage device 63 is an HDD (Hard Disk Drive), semiconductor memory, such as RAM (Random Access Memory) and ROM (Read Only Memory), and the like. The operation console 6 may have all of the HDD, RAM, and ROM as the storage device 63.

The computational processing apparatus 64 is a processor such as a CPU (central processing unit).

The operation console 6 may be configured to be connected with an external storage medium 90. The external storage medium 90 is a non-transitory storage medium having portability, such as a CD (Compact Disk), a DVD (Digital Versatile Disk), USB (Universal Serial Bus) memory, or a hard disk, for example.

As shown in FIG. 1, a direction of the body axis of the subject 5, i.e., a direction of transportation of the subject 5 by the imaging table 4, will be referred to herein as z-direction. Moreover, a vertical direction will be referred to as y-direction, and a horizontal direction orthogonal to the y- and z-directions as x-direction.

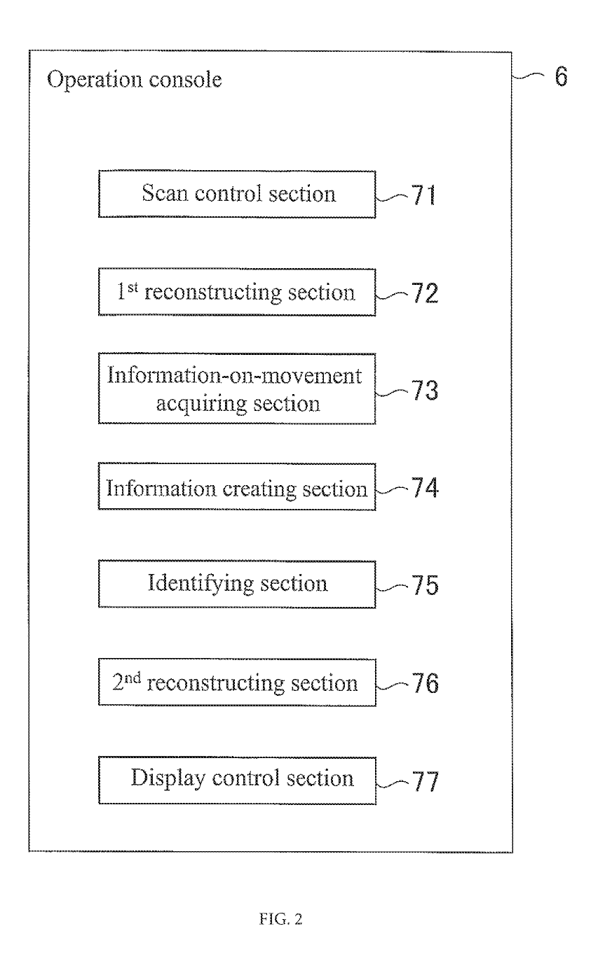

Referring to FIG. 2, the operation console 6 has its functional blocks including a scan control section 71, a first reconstructing section 72, an information-on-movement acquiring section 73, an information creating section 74, an identifying section 75, a second reconstructing section 76, and a display control section 77. The computational processing apparatus 64 executes functions of the scan control section 71, first reconstructing section 72, information-on-movement acquiring section 73, information creating section 74, identifying section 75, second reconstructing section 76, and display control section 77 by prespecified programs. The prespecified programs are stored in, for example, a non-transitory storage medium, such as the HDD or ROM, constituting the storage device 63. The programs may also be stored in the non-transitory external storage medium 90 that is externally connected.

The scan control section 71 controls the gantry/table control section 30 in response to an operation by the operator so that a scan is performed for a required range in the subject in its body-axis direction (z-direction). In the present embodiment, the required range in the subject is a heart. In the present embodiment, a helical scan is performed as the scan, and data at a plurality of slice positions in the subject are collected. The scan control section 71 is an exemplary embodiment of the control section in the present invention.

The first reconstructing section 72 reconstructs a plurality of temporally different first radiation tomographic images for required slice positions in the subject based on the data acquired by scanning the required range in the subject with X-rays. Details thereof will be discussed later. The first reconstructing section 72 is an exemplary embodiment of the first reconstructing section in the present invention.

The information-on-movement acquiring section 73 detects information on movement of a body part in the subject based on the plurality of first radiation tomographic images obtained by the first reconstructing section 72. Details thereof will be discussed later. The information-on-movement acquiring section 73 is an exemplary embodiment of the information-on-movement acquiring section in the present invention.

The information creating section 74 creates information on a temporal change indicating a temporal change of the information on movement acquired by the information-on-movement acquiring section 73. The information creating section 74 is an exemplary embodiment of the information creating section in the present invention.

The identifying section 75 identifies a time when the motion stops or is smaller than a predetermined amount based on the information on a temporal change created by the information creating section 74. The identifying section 75 is an exemplary embodiment of the identifying section in the present invention.

The second reconstructing section 76 reconstructs a second radiation tomographic image for the subject at the aforementioned time identified by the identifying section 75. The second reconstructing section 76 is an exemplary embodiment of the second reconstructing section in the present invention.

The display control section 75 controls the display device 62 to display several kinds of images and text on its screen.

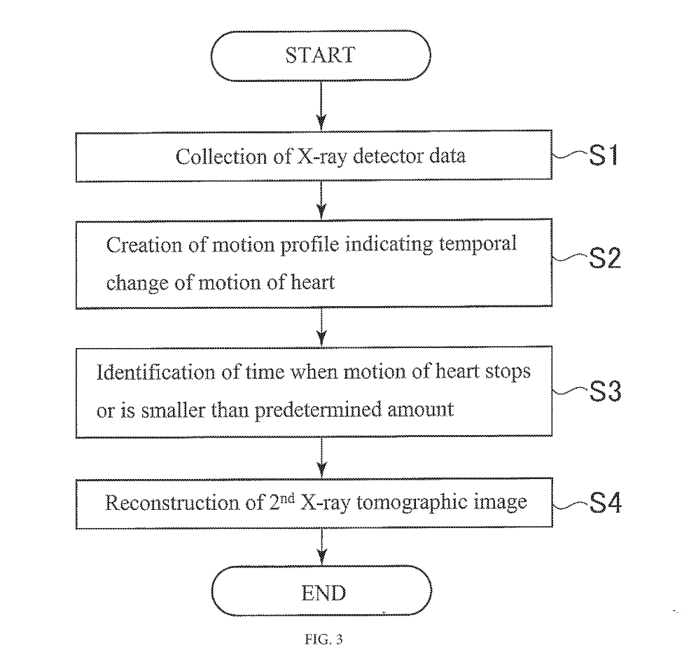

Next, the flow of processing in the X-ray CT system in accordance with the present embodiment will be described based on the flow chart in FIG. 3. First, at Step S1, a scan is performed and X-ray detector data is collected. Specifically, the scan control section 71 controls the gantry/table control section 30 to perform a helical scan on a body part to be imaged in a subject, which is an object of interest to be imaged. The body part to be imaged is a heart of the subject. The helical scan is achieved by emitting X-rays from an X-ray focus of the X-ray tube 21 onto the subject while rotating the X-ray tube 21 and X-ray detector 24 around the subject and at the same time horizontally translating the cradle 41. The X-ray tube 21 and X-ray detector 24 are rotated n (n≧2) times around the subject. Thus, X-ray detector data are collected in a plurality of views along a helical axis.

Next, at Step S2, the information creating section 74 creates a motion profile indicating a temporal change of motion of the heart. The motion profile is an exemplary embodiment of the information on a temporal change indicating a temporal change of the information on movement of the body part in the subject.

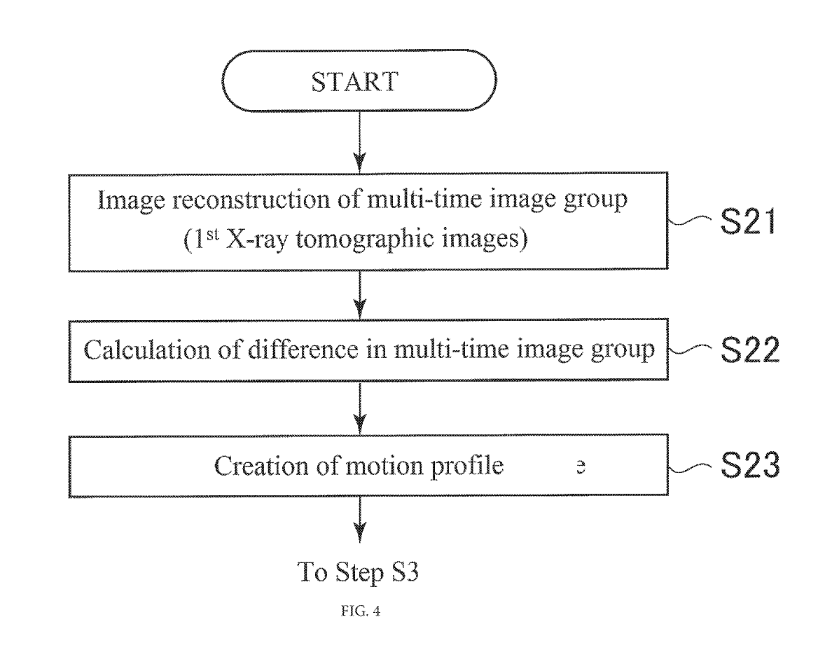

Now the processing at Step S2 will be described in detail based on the flow chart in FIG. 4. At Step S21 in FIG. 4, image reconstruction for a multi-time image group is performed. Specifically, the first reconstructing section 72 first applies pre-processing to the X-ray detector data in a plurality of views along a helical axis described earlier to obtain projection data in the plurality of views. The projection data is then multiplied by a required weighting function, and back-projected to thereby reconstruct a multi-time image group comprised of an image with no time shift and images with forward and backward time shifts. The multi-time image group is reconstructed for each of the plurality of slice positions. The images (in the multi-time image group) reconstructed at Step S21 will be referred to as first X-ray tomographic images herein.

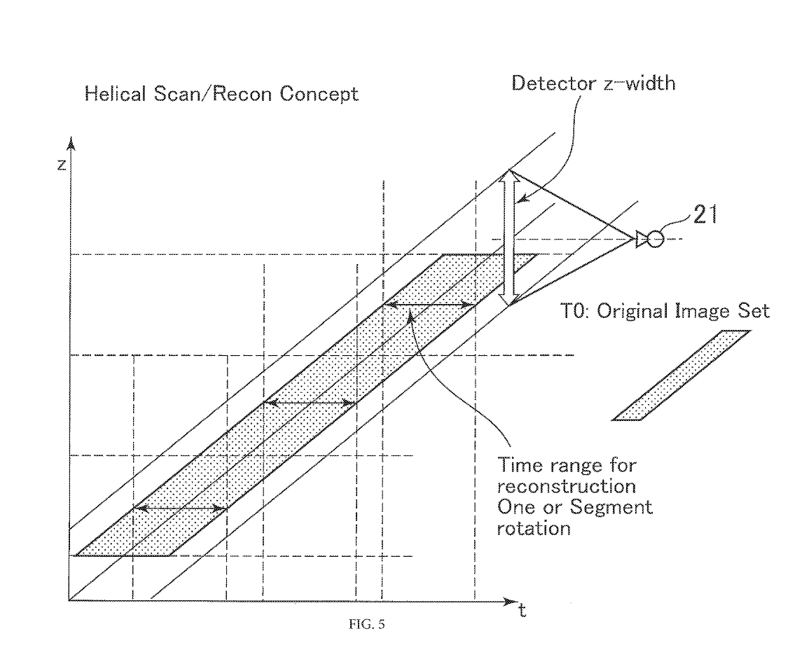

The reconstruction for a multi-time image group will now be described in detail. In general, a time and a position are in a one-to-one relationship in helical image reconstruction. FIG. 5 shows a relationship between time t and position z during imaging according to a general helical scan. Because of helical imaging, the position of the X-ray detector changes with the lapse of time in one-to-one correspondence. A time and an image position are also in one-to-one correspondence because the position of a produced image is always created at the center of a range of detector movement around a specific time T0. Note that for a required amount of data to produce an image (an amount represented by Time range in the figure), data for one rotation of the gantry (to be precise, for one rotation plus the fan angle of the detector) or data for a half rotation (to be precise, for a half rotation plus the fan angle of the detector) are required.

In helical cardiac imaging, consistent image reconstruction at a specific time is required.

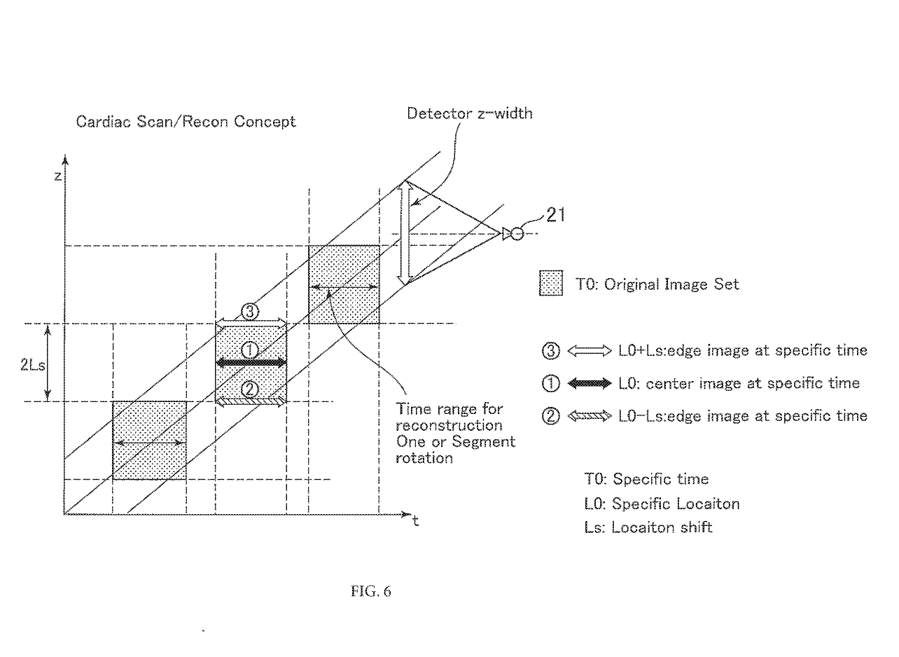

FIG. 6 shows a relationship between time t and position z in helical cardiac imaging. Because of helical imaging, the position of the X-ray detector changes with the lapse of time in one-to-one correspondence. However, data used in image reconstruction at a specific time T0 has a margin for a location shift in the z-axis direction taking account of the z-width of the X-ray detector. In cardiac image reconstruction, this may be used to create a group of cardiac images that are stationary at the specific time T0 by producing images at a plurality of positions at the specific time T0. FIG. 6 shows a case in which images are produced at three mutually different positions L0, L0−Ls, L0+Ls, respectively, to create an image group of the three images. Representing the positional width here as 2Ls (2×location shift), a central image in the image group is an image with no location shift (whose position* is L0), while images at ends in position are images with location shifts by Ls in positive and negative directions.

On the other hand, the data used in image reconstruction at a specific position sometimes has a margin for a time shift in a temporal-axis direction taking account of the z-width of the X-ray detector. Especially in cardiac imaging, there is a sufficient margin because the helical pitch is low. This may be used to produce images at a plurality of times at the specific position.

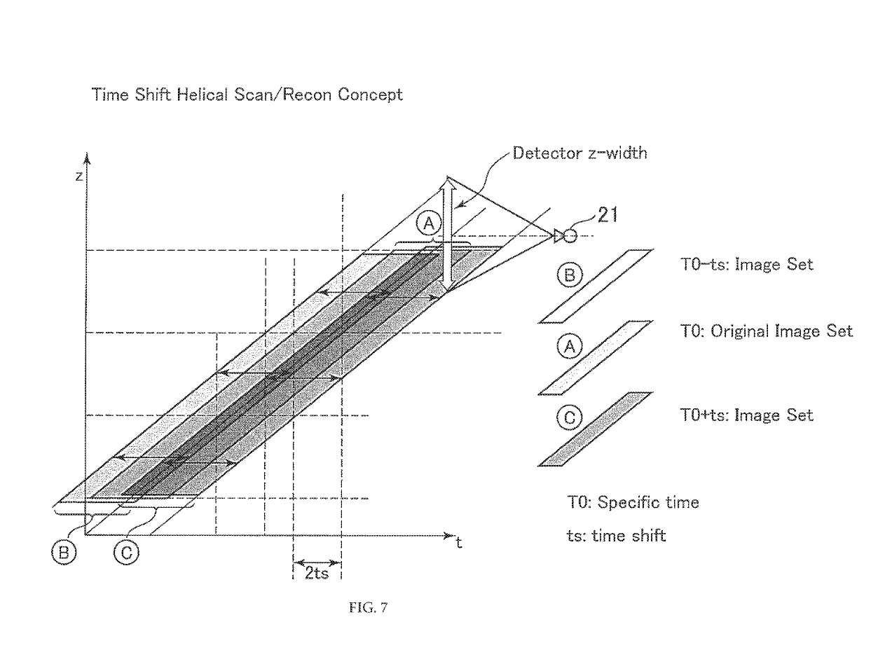

FIG. 7 shows the concept of reconstructing an image temporally shifted by the margin described above. The time shift is equivalent to the location shift considering the relationship between time t and position z. Comparing in parallel an image produced at a specific time T0 with a location shift with an image produced in another time zone at the same position without a location shift, they may be considered to be images with a time shift in spite of the fact that they are images at the same position because the time of acquisition of data is different. Hence, it is possible to apply a time shift within a range of valid data in the detector as shown. FIG. 7 shows a case in which images are produced at three mutually different times T0, T0−ts, T0+ts, respectively, to create an image group of these three images. Representing the temporal width here as 2ts (2×time shift), a central image in the image group is an image with no time shift (whose time is T0), while images at ends in time are images with a time shift by is in positive and negative directions. The first reconstructing section 72 produces an image with no time shift (whose time is T0) and two images (whose time is T0−ts and T0+ts) shifted forward and backward, by image reconstruction. Thus, the first reconstructing section 72 produces each of a plurality of temporally different images by image reconstruction based on data in each of a plurality of temporally different ranges at a required slice position.

A set of the image with no time shift and the two images temporally shifted forward and backward described above is referred to as a multi-time image group. The first reconstructing section 72 produces a multi-time image group for each of a plurality of slice positions in a range to be imaged (required range) in the subject. Each of images, i.e., first X-ray tomographic images, constituting the multi-time image group is an exemplary embodiment of the first radiation tomographic image in the present invention.

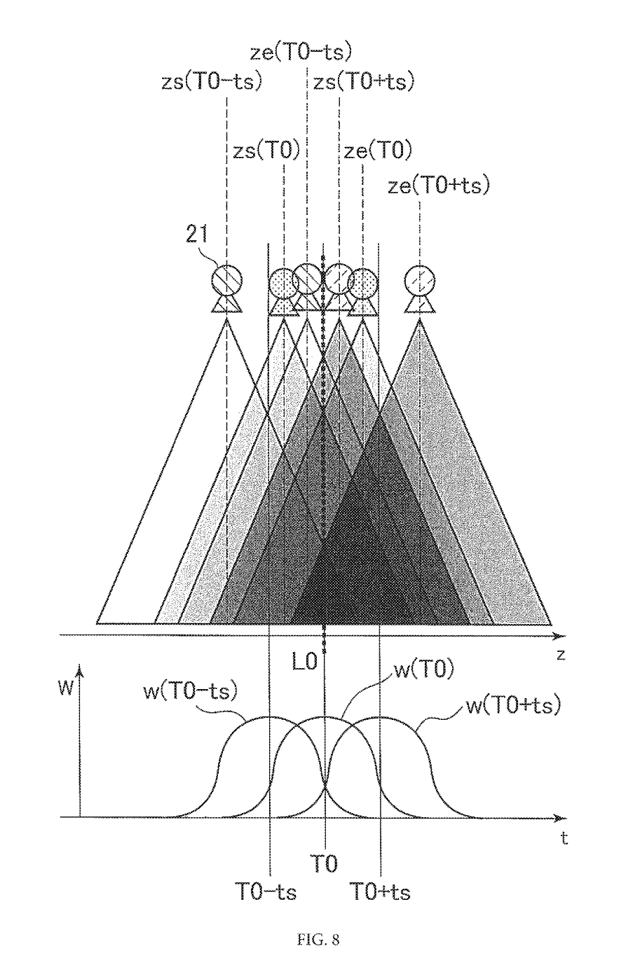

Next, a weighting function used in reconstruction of a multi-time image group will be described. FIG. 8 shows depiction of a relationship between a position of a helical image and a position of the X-ray detector during data collection when a time shift is applied, and a temporal position of a weighting function and a profile thereof.

FIG. 8 represents a general case of a positional relationship between temporally forward and backward data regions in the X-ray detector, and a shift of the weighting function in temporal position. In FIG. 8, a physical position L0 indicated by a dashed line represents a slice position to which image reconstruction is to be applied, the slice position corresponding to time T0. Moreover, two positions zs(T0) and ze(T0) of the X-ray detector represent a position of the start of collection and a position of the end of collection of data used in reconstruction of an image with no time shift. Likewise, positions zs(T0−ts) and ze(T0−ts) represent a position of the start of collection and a position of the end of collection of data used in reconstruction of an image temporally shifted to the negative side. Positions zs(T0+ts) and ze(T0+ts) represent a position of the start of collection and a position of the end of collection of data used in reconstruction of an image temporally shifted to the positive side. A weighting function w(T0) is a weighting function superposed on the data used in reconstruction of the image with no time shift. A weighting function w(T0−ts) is a weighting function superposed on the data used in reconstruction of the image temporally shifted to the negative side. A weighting function w(T0+ts) is a weighting function superposed on the data used in reconstruction of the image temporally shifted to the positive side.

When a time shift is applied, the physical position of the X-ray detector, more particularly, a central position of the data region used in image reconstruction, coincides with the time of the image, but does not coincide with the position of the image. Moreover, the weighting function w(T0) here is shifted in the temporal-axis direction to become w(T0−ts) and w(T0+ts), although its profile shape is not modified according to the time shift in FIG. 8. However, when a time shift is applied and geometrically farther data is used, the cone angle of an X-ray path for the data is increased, and it is geometrically expected to cause increased artifacts. On the other hand, a conjugate beam opposite at a rotation angle different by 180 degrees may have corresponding data with a smaller cone angle.

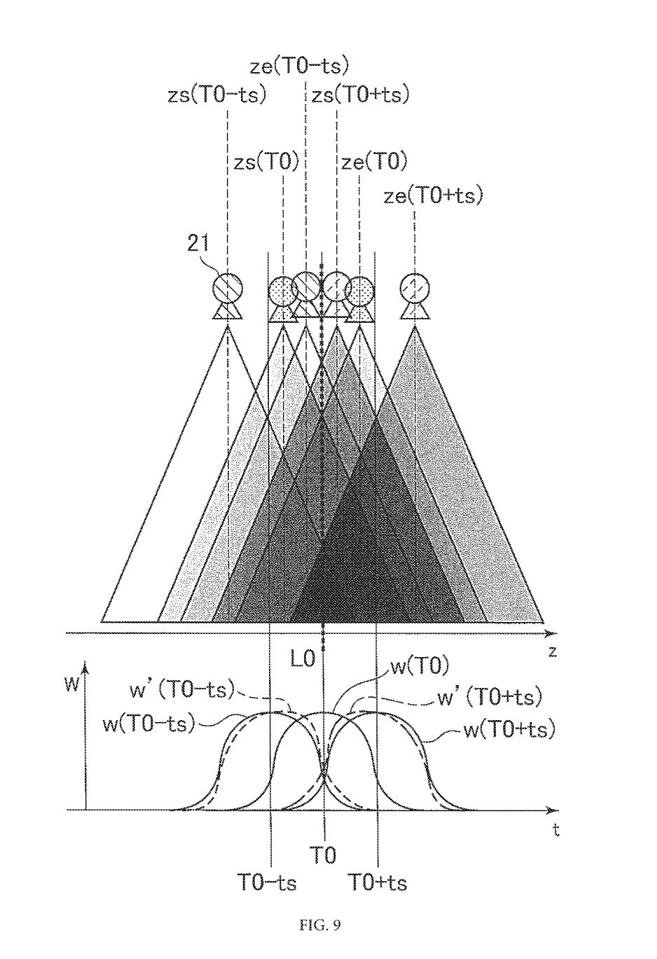

FIG. 9 shows an example of a modified profile of the weighting function when a time shift is applied. As shown, a modified weighting function taking account of the cone angle of an X-ray path for data to be used according to the time shift ±ts as described above is consequently able to reduce cone-beam artifacts more. In other words, the time T0±ts corresponding to the middle of the profile of a time-shifted weighting function coincides with the center of a period of collection of the data used in image reconstruction, so that a weighting function w′(T0±ts) formed by reducing the weight for a region having a larger cone angle of the X-ray path by the time shift while increasing the weight for a region having a smaller cone angle of the X-ray path may be used to reduce cone-beam artifacts more than the case in which a simple time shift is applied. Thus, it is important to modify the shape of a weighting function according to a time shift, in addition to a simple time shift of the weighting function. At that time, the profile of the weighting function may be modified to have a half width as equal as possible between temporally front and back sides to minimize the impact on the amount of a time shift.

Once a multi-time image group has been obtained for each of the plurality of slice positions at Step S21, the information-on-movement acquiring section 73 calculates a difference in the multi-time image group at Step S22. The difference is calculated for each of the plurality of slice positions. The difference may be calculated for a plurality of different times at one slice position.

Now calculation of the aforementioned difference will be particularly described. The information-on-movement acquiring section 73 first divides each of images constituting a multi-time image group into a plurality of local images. Next, the information-on-movement acquiring section 73 calculates, for each combination of local images at the same position in the subject but at different times, a difference value between local images in the combination.

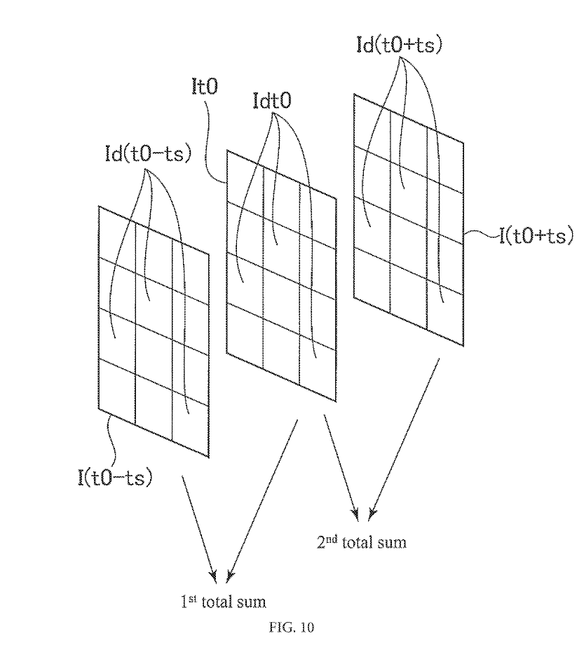

The calculation of the difference value will be described in more detail. For example, as shown in FIG. 10, the information-on-movement acquiring section 73 takes a difference between corresponding pixels in each of local images Idt0 in an image It0 at time T0, i.e., an image It0 with no time shift, and in each of local images Id(t0−ts) in an image I(t0−ts) at time T0−ts, i.e., a temporally forward time-shifted image I(t0−ts), and calculates a total sum of absolute values of the difference values within the local image. The information-on-movement acquiring section 73 then calculates a sum of all of the total sums each obtained in each of the plurality of local images as a first total sum. Likewise, the information-on-movement acquiring section 73 takes a difference between corresponding pixels in each of the local images Idt0 in the image It0 with no time shift, and in each of local images Id(t0+ts) in an image I(t0+ts) at time T0+ts, i.e., a temporally backward time-shifted image I(t0+ts), and calculates a total sum of absolute values of the difference values within the local image. The information-on-movement acquiring section 73 then calculates a sum of all of the total sums each obtained in each of the plurality of local images as a second total sum.

Note that the total sum obtained in each of the plurality of local images is an exemplary embodiment of the difference value between local images in the combination.

The information-on-movement acquiring section 73 calculates a feature quantity using the first total sum and second total sum as the aforementioned difference. For example, the information-on-movement acquiring section 73 may calculate a total difference value obtained by adding the first total sum and second total sum together as the aforementioned difference. Alternatively, it may calculate an average value of the first total sum and second total sum as the aforementioned difference. The total difference value or average value is calculated for each of the plurality of slice positions. It should be noted that the aforementioned difference is not limited to the total difference value or average value.

The total difference value and average value constitute an exemplary embodiment of the information on movement in a whole of the first radiation tomographic image.

The information-on-movement acquiring section 73 may also calculate an index value as the aforementioned difference based on the total difference value or average value using a required formula. In the present embodiment, a larger index value indicates a larger value of the aforementioned difference and a greater amount of movement.

It should be noted that the information-on-movement acquiring section 73 may take a difference between corresponding pixels (pixels whose positions are the same in the subject) in the image with no time shift and in the temporally forward time-shifted image without dividing the images into local images, and calculate a total sum thereof as the first total sum. Likewise, the information-on-movement acquiring section 73 may take a difference between corresponding pixels in the image with no time shift and in the temporally backward time-shifted image without dividing the images into local images, and calculate a total sum thereof as the second total sum.

Here, the aforementioned difference is larger as more motion of the heart is present between the image with no time shift and temporally shifted image, while it is smaller as less motion of the heart is present. Therefore, the aforementioned difference is an exemplary embodiment of the information on movement of the body part in the subject in the present invention.

Next, at Step S23, the information creating section 74 creates a motion profile based on the difference obtained at Step S22. Specifically, the information creating section 74 first plots the differences for a plurality of different times obtained at Step S22 against the temporal axis. The differences for a plurality of different times are differences obtained at each of a plurality of slice positions. The information creating section 74 plots the differences at times with no time shift described above (T0 described above, for example).

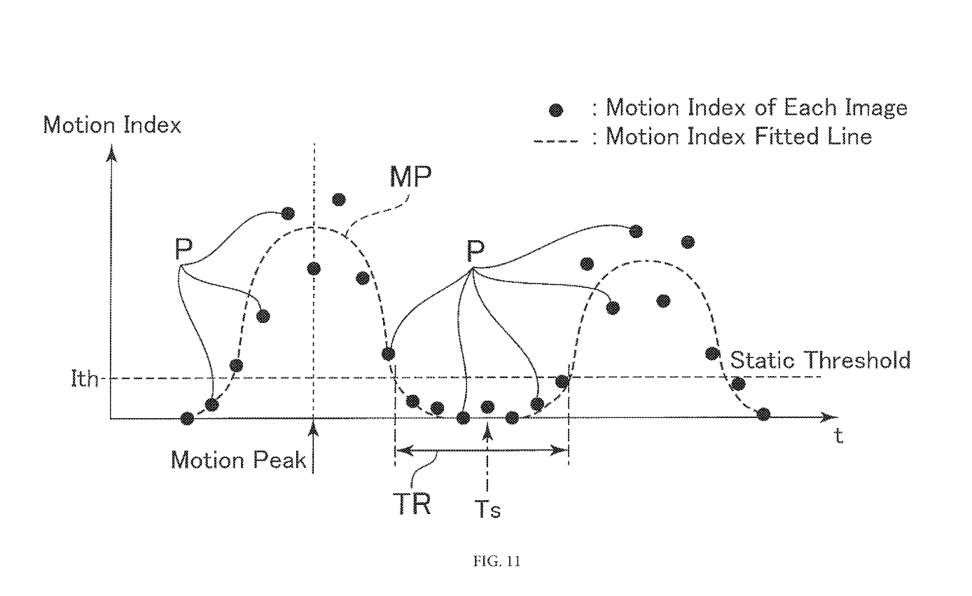

For example, the information creating section 74 plots the index values against the temporal axis. FIG. 11 shows index values plotted for a plurality of different times in a coordinate formed by the time (t) in a horizontal axis and the index value (Motion Index) in a vertical axis. A point marked by symbol P indicates an index value at a certain time. The certain time refers to a time (T0) with no time shift, for example.

Next, the information creating section 74 creates a motion profile MP indicated by a dashed line in FIG. 11 by applying fitting to a plurality of the points P. The motion profile MP is a curve representing a temporal change of the index value. It should be noted that the motion profile MP comprises index values at a plurality of slice positions. The motion profile MP is an exemplary embodiment of the information on a temporal change in the present invention.

The motion profile obtained at Step S2 may be displayed on the display device 62.

Once the motion profile has been obtained at Step S2 as described above, the identifying section 75 identifies a time when motion of the heart stops or is smaller than a predetermined amount based on the motion profile at Step S3.

For example, the identifying section 75 identifies a time when the index value is equal to or lower than a predefined threshold in the motion profile to identify the aforementioned time. The following description will be made exemplifying the motion profile MP shown in FIG. 11, wherein a range indicated by symbol TR represents a time zone in which the index value is equal to or lower than a predefined threshold Ith. After the identifying section 75 has identified the time zone TR in the motion profile MP, it identifies a time Ts at a central position of the time zone TR as the aforementioned time. Here, the time Ts is a time when motion of the heart momentarily stops. The technique of identifying the aforementioned time is exemplary and is not limited thereto. The identifying section 75 may identify a time when motion of the heart is smaller than a predetermined amount.

A portion (upward-convex portion) in the motion profile MP in which the index value is at a local maximum is a portion in which motion of the heart is at its peak. On the other hand, a portion (downward-convex portion, the time Ts) in which the index value is at a local minimum in the motion profile MP is a portion in which motion of the heart momentarily stops.

While only one time zone TR is shown in FIG. 11, the identifying section 75 may identify a plurality of the time zones TR. In this case, the identifying section 75 identifies the time Ts for each of the plurality of times zones TR. Thus, a plurality of the aforementioned times may be identified by the identifying section 75.

As for the heart, its motion momentarily stops at diastole and systole. The diastole and systole are alternately repeated. The identifying section 75 identifies whether the aforementioned time identified in the motion profile is in diastole or systole based on, for example, data of the image produced by the first reconstructing section 72.

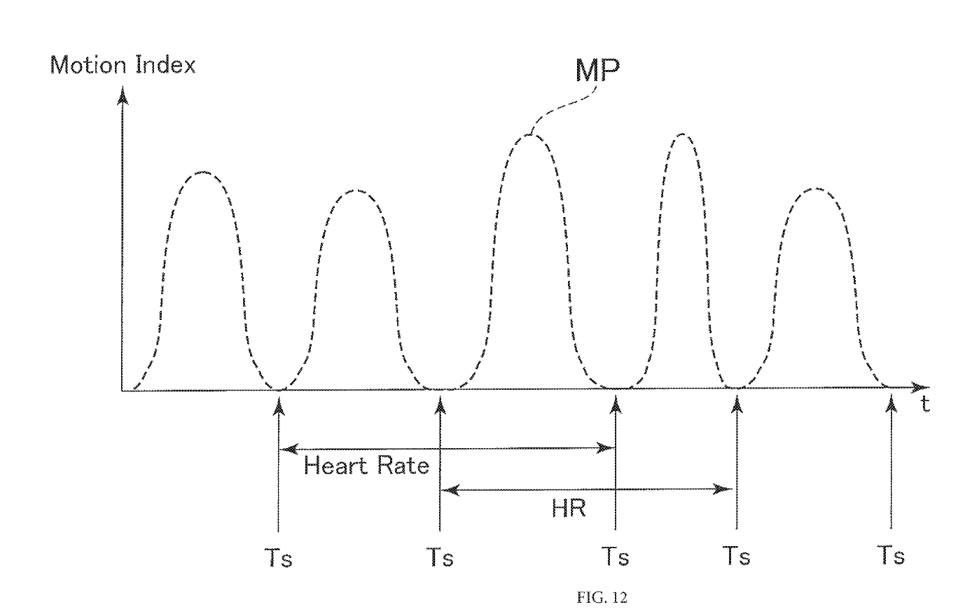

More specifically, a plurality of times Ts are identified as the aforementioned time in a motion profile MP shown in FIG. 12. The identifying section 75 compares regions of air in data at a pair of times Ts adjacent to each other among the plurality of times Ts. Here, regions of air exist around the heart, where the regions of air expand more in systole than in diastole. Therefore, the identifying section 75 recognizes one of the pair of times Ts of interest to be compared that has greater regions of air in data of the image produced by the first reconstructing section 72 as systole, and the other as diastole. Since the systole and diastole alternately occur, the identifying section 75 identifies, based on the identification of systole and diastole for one pair of times Ts, systole and diastole for the others of the plurality of times Ts.

Next, at Step S4, the second reconstructing section 76 reconstructs a second X-ray tomographic image for the subject at the aforementioned time identified at Step S3. Since systole and diastole are distinguished from each other as the aforementioned time, a second X-ray tomographic image at systole and that in diastole are obtained at Step S4 here.

This will be described in more detail. The second reconstructing section 76 reconstructs a second X-ray tomographic image at the time identified at Step S3 based on the data collected at Step S1 and obtained at that time.

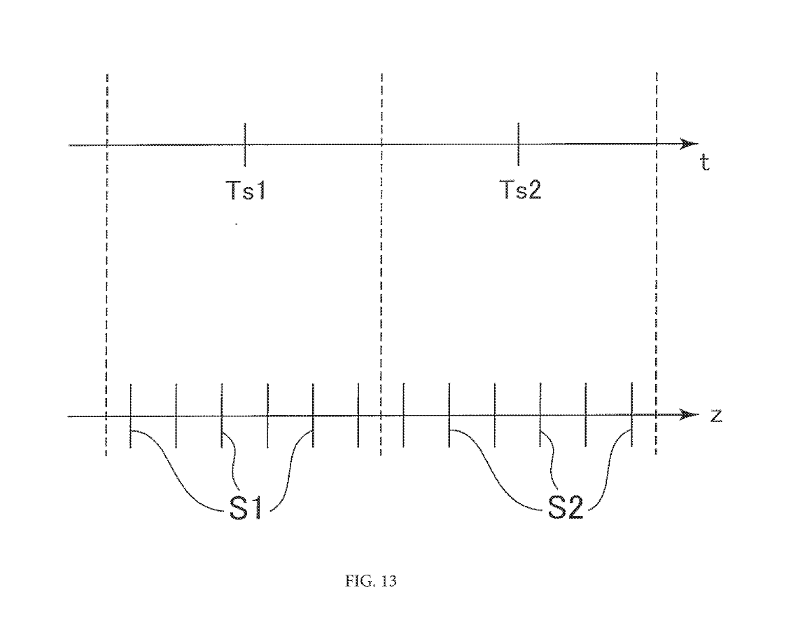

The second reconstructing section 76 reconstructs second X-ray tomographic images for a plurality of slice positions at one time. This will be particularly described based on FIG. 13. In FIG. 13, times Ts1, Ts2 are shown as the time identified at Step S3. The times Ts1, Ts2 are mutually different times. The second reconstructing section 76 reconstructs second X-ray tomographic images for a plurality of slice positions S1 at time Ts1 based on data obtained at the time Ts1. The plurality of slice positions S1 are mutually different positions in the body-axis direction of the subject. The second reconstructing section 76 also reconstructs second X-ray tomographic images for a plurality of slice positions S2 at time Ts2 based on data obtained at the time Ts2. The plurality of slice positions S2, again, are mutually different positions in the body-axis direction of the subject. The plurality of slice positions S1 and plurality of slice positions S2 are also mutually different positions in the body-axis direction of the subject.

It should be noted that the data obtained at the times Ts1, Ts2 are data over a predefined temporal width around the times Ts1, Ts2. The predefined temporal width is a temporal width in which data required to produce one image are collected.

The second reconstructing section 76 reconstructs the second X-ray tomographic images in a region of the whole heart, which is the body part to be imaged. Thus, second X-ray tomographic images at the time of diastole and those at the time of systole are obtained.

According to the present embodiment described above, an X-ray tomographic image at a time when motion of the heart stops or is small may be obtained without using an EKG signal while suppressing the number of X-ray tomographic images to produce.

Next, a variation of the embodiment will be described. In the variation, the information creating section 74 may create a motion profile on a local image-by-local image basis. This will be described in detail. A difference is taken between corresponding pixels in each of local images Idt0 in an image It0 with no time shift and in each of local images Id(t0−ts) in a temporally forward time-shifted image I(t0−ts) to calculate a difference value, and a total sum of absolute values of the difference values within the local image is taken as a third total sum. That is, the third total sum is a total sum of absolute values of difference values obtained in each of the local images. Moreover, a difference is taken between corresponding pixels in each of the local images Idt0 in the image It0 with no time shift and in each of local images Id(t0+ts) in a temporally backward time-shifted image (t0+ts) to calculate a difference value, and a total sum of absolute values of the difference values within the local image is taken to as a fourth total sum. That is, the fourth total sum is also a total sum of absolute values of difference values obtained in each of the local images.

The information-on-movement acquiring section 73 calculates a feature quantity using the third total sum and fourth total sum, in place of the first total sum and second total sum, as the aforementioned difference at Step S22 described above. For example, the information-on-movement acquiring section 73 may calculate a total difference value in which the third total sum and fourth total sum are added together as the aforementioned difference between corresponding local images. The total difference value here is obtained on a local image-by-local image basis. Alternatively, the information-on-movement acquiring section 73 may calculate an average value of the third total sum and fourth total sum as the aforementioned difference between corresponding local images. The average value here, again, is obtained on a local image-by-local image basis. Note that the total difference value and average value here are calculated for each of a plurality of slice positions, as in the embodiment described earlier.

The information creating section 74 creates a motion profile for each local image by creating a motion profile based on the feature quantity calculated using the third total sum and fourth total sum.

In the case that the motion profile is created on a local image-by-local image basis, the identifying section 75 identifies a time when motion of the heart stops or is smaller than a predetermined amount on a local image-by-local image basis based on the motion profile at Step S3 described earlier. Then, at Step S4 described earlier, the second reconstructing section 76 reconstructs a partial second X-ray tomographic image at the aforementioned time for each part corresponding to the local image, and then, produces one second X-ray tomographic image comprised of the partial second X-ray tomographic images for a required slice position. It should be noted that the partial second X-ray tomographic image is also obtained at one time for a plurality of slice positions.

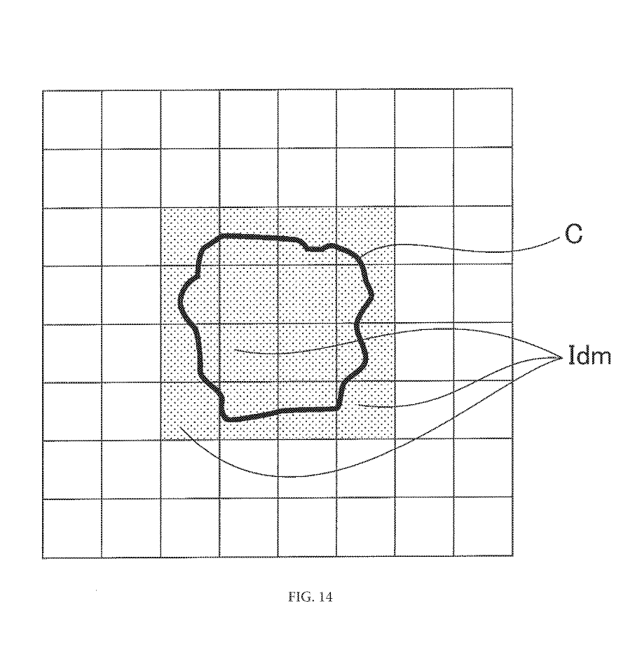

When the motion profile is created on a local image-by-local image basis as described above, the identifying section 75 may identify a local image Idm in which motion of the heart is detected. A region hatched by dots in FIG. 14 indicates local images Idm in which motion of the heart is detected. It should be noted that in FIG. 14, symbol C designates a contour of the heart.

The identifying section 75 detects motion of the heart based on the motion profile. The identifying section 75 identifies that the heart is in motion when the index value is greater than a predefined threshold Ith in the motion profile, for example.

The identifying section 75 may identify that the heart is in motion when the index value is greater than the predefined threshold Ith only in the case that the waveform of the motion profile is periodic. It may also detect motion of the heart at a plurality of different times, and identify a region of the local images Idm at each time.

The identifying section 75 may identify the region of the local images Idm in which motion of the heart is detected as a region of the heart, and identify whether the aforementioned time identified in the motion profile is in diastole or systole based on the size of the region of the heart.

In the case that a region of the heart is identified as described above, regions of air used for identifying diastole and systole in the embodiment described earlier may be identified within the region of the heart.

The display control section 77 may display an image Ic indicating that motion of the heart is detected on the display device 62, although not particularly shown. The display control section 77 may display the image in the first X-ray tomographic image I1, for example. The image Ic is a color image through which a background black-and-white image passes, for example. The image Ic is displayed in a portion in the first X-ray tomographic image I1 corresponding to local images in which motion of the heart is detected by the information creating section 74, for example.

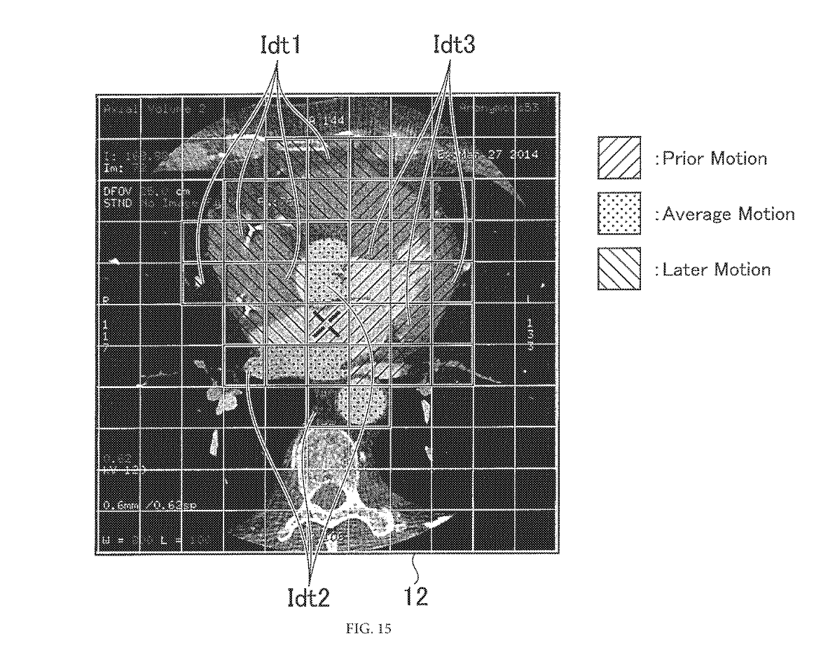

The display control section 77 may display an image Idt indicating a temporal difference in motion of the heart, as shown in FIG. 15, based on a difference in phase of the waveform of the motion profile among a plurality of local images. The display control section 77 displays the image Idt in the second X-ray tomographic image I2, for example, displayed on the display device 62.

The display control section 77 displays a first image Idt1, a second image Idt2, and a third image Idt3 as the image Idt. The first image Idt1 indicates a region having a time at which motion of the heart starts from its momentary stop later than the second image Idt2. The third image Idt3 indicates a region having a time at which motion of the heart starts from its momentary stop earlier than the second image Idt2.

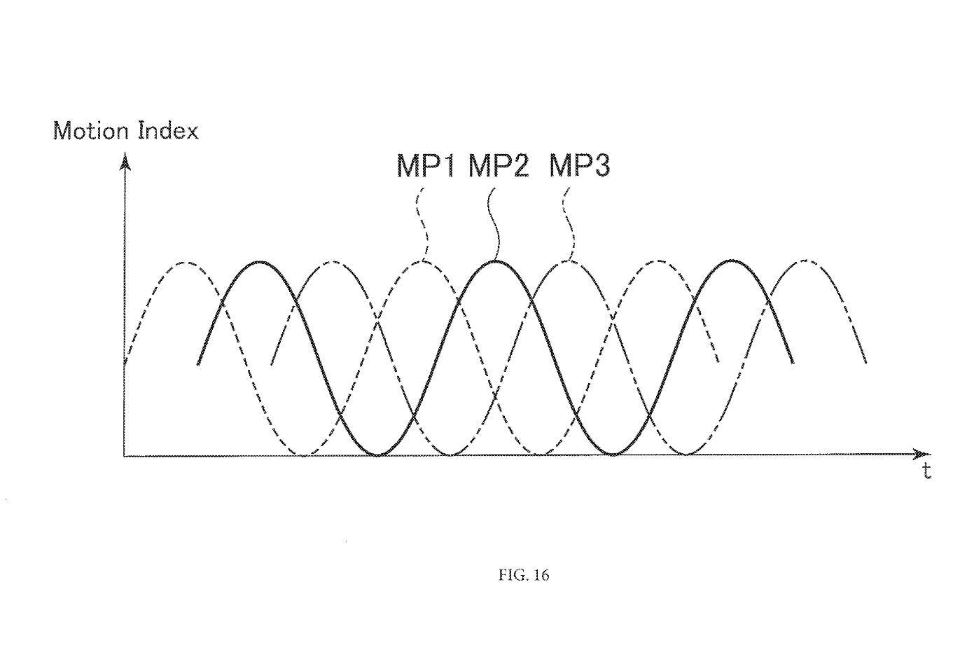

The first image Idt1 is displayed in a portion in the second X-ray tomographic image I2 corresponding to local images having a first motion profile MP1 shown in FIG. 16, for example. The second image Idt2 is displayed in a portion in the second X-ray tomographic image I2 corresponding to local images having a second motion profile MP2 shown in FIG. 16, for example. The third image Idt3 is displayed in a portion in the second X-ray tomographic image I2 corresponding to local images having a third motion profile MP3 shown in FIG. 16, for example.

The first motion profile MP1 has a waveform whose phase is behind that of the second motion profile MP2. The third motion profile MP3 has a waveform whose phase is in advance of that of the second motion profile MP2.

The first image Idt1, second image Idt2, and third image Idt3 are displayed with mutually different display patterns. While in FIG. 15, the first image Idt1 and second[sic] image Idt3 are hatched with oblique stripes in mutually different directions and the second image Idt2 is hatched with dots, the first image Idt1, second image Idt2, and third image Idt3 may be color images in mutually different colors through which a background black-and-white image (second X-ray tomographic image I2) passes, for example.

While the present invention has been described with reference to the embodiments, it will be easily recognized that the invention may be practiced with several modifications without changing the spirit and scope thereof. For example, a case in which three images are produced as a multi-time image group is described in the embodiment above, it is sufficient that the multi-time image group is comprised of at least two images.

Moreover, the technique of identifying a time by the identifying section 75 described in the embodiment above is exemplary and is not limited to that described above.

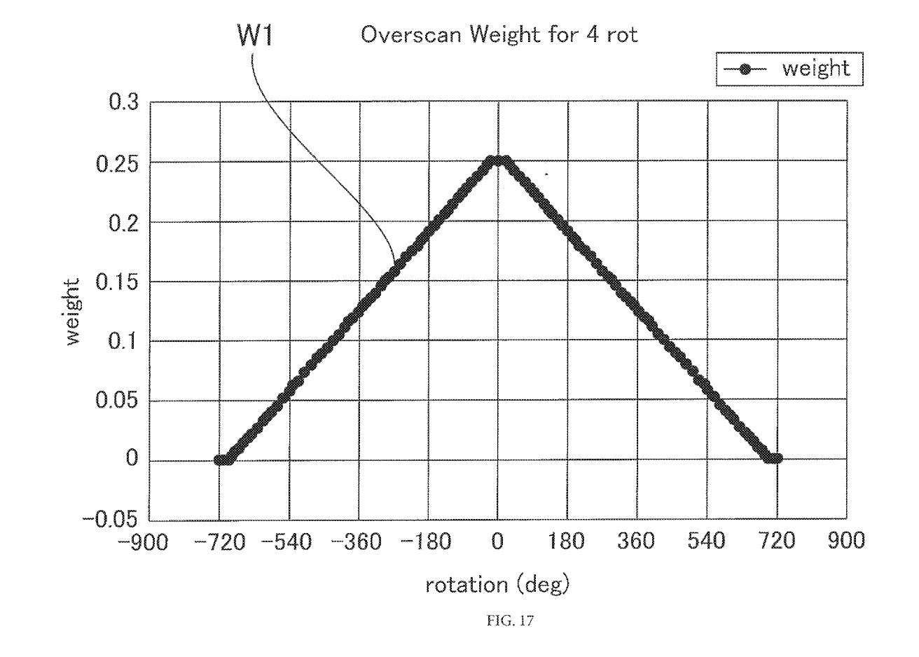

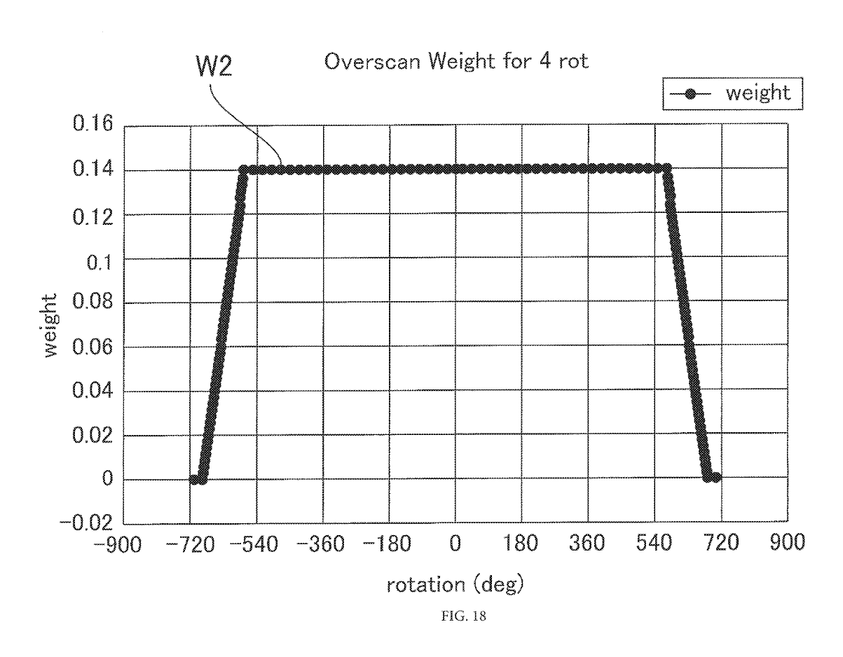

Furthermore, the weighting function described in the embodiment above is exemplary and is not limited to that described above. For example, the weighting function may be a weighting function W1 shown in FIG. 17 or a weighting function W2 shown in FIG. 18.

While the present embodiment is an X-ray CT apparatus, the invention is also applicable to tomographic imaging apparatuses using radiation other than X-rays, for example, those using gamma rays.

In addition, a program for causing a computer to function as several means for performing control and/or processing in the X-ray CT apparatus described above and a recording medium in which such a program is stored each constitute an exemplary embodiment of the invention as well.

We claim:

1. A radiation tomographic imaging apparatus characterized in comprising:
a first reconstructing section for reconstructing a plurality of temporally different first radiation tomographic images for a required slice position in a subject based on data obtained by scanning a required range in said subject in its body-axis direction with radiation;
an information-on-movement acquiring section for acquiring information on movement of a body part in said subject in said required range based on said plurality of first radiation tomographic images;
information creating section for creating information on a temporal change indicating a temporal change of said information on movement acquired by said information-on-movement acquiring section;
an identifying section for identifying a time when motion of said body part in said subject stops or said motion is smaller than a predetermined amount based on said information on a temporal change; and
a second reconstructing section for reconstructing a second radiation tomographic image for said subject at said time based on said data.

2. The radiation tomographic imaging apparatus as recited in claim 1, characterized in that: said body part in said subject is a heart.

3. The radiation tomographic imaging apparatus as recited in claim 1, characterized in that: said second reconstructing section reconstructs the second radiation tomographic image for said subject at said time for a plurality of slice positions based on the data obtained at said time identified by said identifying section.

4. The radiation tomographic imaging apparatus as recited in claim 2, characterized in that: said second reconstructing section reconstructs the second radiation tomographic image for said subject at said time for a plurality of slice positions based on the data obtained at said time identified by said identifying section.

5. The radiation tomographic imaging apparatus as recited in claim 1, characterized in comprising:
a control section for controlling a data collection chain including a multi-slice detector to perform a helical scan as said scan and collect the data in said required range in said subject in its body-axis direction, wherein
said first reconstructing section reconstructs each of said plurality of temporally different first radiation tomographic images at said required slice position based on each of a plurality of temporally different ranges of the data collected by said multi-slice detector.

6. The radiation tomographic imaging apparatus as recited in claim 2, characterized in comprising:
a control section for controlling a data collection chain including a multi-slice detector to perform a helical scan as said scan and collect the data in said required range in said subject in its body-axis direction, wherein
said first reconstructing section reconstructs each of said plurality of temporally different first radiation tomographic images at said required slice position based on each of a plurality of temporally different ranges of the data collected by said multi-slice detector.

7. The radiation tomographic imaging apparatus as recited in claim 3, characterized in comprising:
a control section for controlling a data collection chain including a multi-slice detector to perform a helical scan as said scan and collect the data in said required range in said subject in its body-axis direction, wherein
said first reconstructing section reconstructs each of said plurality of temporally different first radiation tomographic images at said required slice position based on each of a plurality of temporally different ranges of the data collected by said multi-slice detector.

8. The radiation tomographic imaging apparatus as recited in claim 4, characterized in comprising:
a control section for controlling a data collection chain including a multi-slice detector to perform a helical scan as said scan and collect the data in said required range in said subject in its body-axis direction, wherein
said first reconstructing section reconstructs each of said plurality of temporally different first radiation tomographic images at said required slice position based on each of a plurality of temporally different ranges of the data collected by said multi-slice detector.

9. The radiation tomographic imaging apparatus as recited in claim 5, characterized in that:
said first reconstructing section is for reconstructing a plurality of temporally different radiation tomographic images for said required slice position using data obtained by applying weighting to the data collected in said required range depending upon a position in said subject in its body-axis direction.

10. The radiation tomographic imaging apparatus as recited in claim 1, characterized in that: said information-on-movement acquiring section calculates differences among said plurality of first radiation tomographic images as said information on movement.

11. The radiation tomographic imaging apparatus as recited in claim 10, characterized in that: said information-on-movement acquiring section calculates, in said plurality of first radiation tomographic images, a difference value for data between pixels lying at the same position in said subject, and acquires said information on movement in a whole of said first radiation tomographic image.

12. The radiation tomographic imaging apparatus as recited in claim 10, characterized in that: said information-on-movement acquiring section divides each of said plurality of first radiation tomographic images into a respective plurality of local images, and calculates, for each combination of a plurality of local images at the same position in said subject but at different times, a difference value between local images in said combination to acquire said information on movement in each of said plurality of local images.

13. The radiation tomographic imaging apparatus as recited in claim 12, characterized in that:
said information-on-movement acquiring section acquires said information on movement in a whole of said first radiation tomographic image based on said information on movement in each of said plurality of local images, and
said information creating section creates said information on a temporal change in the whole of said first radiation tomographic image based on said information on movement in the whole of said first radiation tomographic image.

14. The radiation tomographic imaging apparatus as recited in claim 12, characterized in that:
said information creating section creates said information on a temporal change in each of said plurality of local images based on said information on movement acquired in each of said plurality of local images,
said identifying section identifies said time for each of said plurality of local images, and
said second reconstructing section reconstructs a partial second X-ray tomographic image at said time for each part corresponding to said local image to reconstruct one said second X-ray tomographic image at a required slice position.

15. The radiation tomographic imaging apparatus as recited in claim 12, characterized in that:
said body part in said subject is a heart, and
said identifying section identifies as a region of the heart a region of local images in which motion of said heart is detected among said plurality of local images based on the information on movement acquired by said information-on-movement acquiring section to identify whether said time is in diastole or systole of the heart based on a size of said region of the heart.

16. The radiation tomographic imaging apparatus as recited in claim 12, characterized in comprising: a display control section for displaying an image indicating a temporal difference in motion of said body part in said subject in each of said plurality of local images based on said information on movement acquired in each of said plurality of local images.

17. The radiation tomographic imaging apparatus as recited in claim 1, characterized in comprising: a display section in which said information created by said information creating section is displayed.

18. The radiation tomographic imaging apparatus as recited in claim 1, characterized in that:
said first reconstructing section reconstructs a plurality of temporally different said first radiation tomographic images for each of a plurality of slice positions in said required range,
said movement detecting section acquires said information on movement for each of said plurality of slice positions, and
said information creating section creates said information on a temporal change including said information on movement at each of said plurality of slice positions.

19. The radiation tomographic imaging apparatus as recited in claim 18, characterized in that:
said identifying section identifies a plurality of times as said time, and
said second reconstructing section reconstructs the second radiation tomographic images for a plurality of mutually different slice positions at each of said plurality of times.

20. A radiation tomographic imaging apparatus characterized in comprising a processor executing by a program:
a first reconstructing function of reconstructing a plurality of temporally different first radiation tomographic images for a required slice position in a subject based on data obtained by scanning a required range in said subject in its body-axis direction with radiation;
an information-on-movement acquiring function of acquiring information on movement of a body part in said subject in said required range based on said plurality of first radiation tomographic images;
information creating function of creating information on a temporal change indicating a temporal change of said information on movement acquired by said information-on-movement acquiring function;
an identifying function of identifying a time when motion of said body part in said subject stops or said motion is smaller than a predetermined amount based on said information on a temporal change; and
a second reconstructing function of reconstructing a second radiation tomographic image for said subject at said time based on said data.
